(19) United States
(12) Patent Application Publication
(10) Pub. No.: US 2005/0209245 A1
(43) Pub. Date: Sep. 22, 2005

(54) Process for preparing N-aryl-piperazine derivatives

(75) Inventors: Anita Wai-Yin Chan, Fort Lee, NJ (US); Jianxin Ren, Nanuet, NY (US); Archana Sharma, Park Ridge, NJ (US)

Correspondence Address: WOODCOCK WASHBURN LLP, ONE LIBERTY PLACE - 46TH FLOOR, PHILADELPHIA, PA, 19103, US

(73) Assignee: Wyeth, Madison, NJ (US)

(21) Appl. No.: 11/082,510

(22) Filed: Mar. 17, 2005

Related U.S. Application Data
(60) Provisional application No. 60/554,666, filed on Mar. 19, 2004.

(51) Int. Cl. A61K 31/496; C07D 43/02

(52) U.S. Cl. 514/253.01; 544/360

(57) ABSTRACT

Processes for preparing N-aryl-piperazine derivatives of formula I 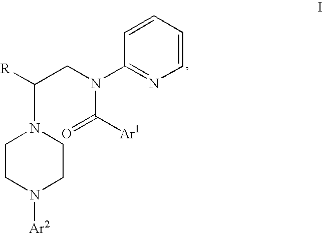 particularly (R)-4-cyano-N-[2-[4-(2,3-dihydro-1,4-benzodioxan-5-yl)-1-piperazinyl-propyl]-N-(2-pyridinyl)-benzamide, are disclosed. Compositions comprising N-aryl-piperazine derivatives and low levels of common impurities are also disclosed. In addition, products produced by the process are disclosed.

### CROSS REFERENCE TO RELATED APPLICATIONS

This application claims the benefit of U.S. application Ser. No. 60/554,666 filed Mar. 19, 2004, the entire disclosure of which is herein incorporated by reference.

### FIELD OF THE INVENTION

The present invention relates to processes for preparing N-aryl-piperazine derivatives, particularly the large-scale production of N-aryl-piperazine derivatives useful, inter alia, as 5-HT_{1A} receptor modulators.

### BACKGROUND OF THE INVENTION

Certain N-aryl-piperazine derivatives possess pharmaceutical activity. In particular, certain N-aryl piperazine derivatives act on the central nervous system (CNS) by binding to 5-HT receptors. In pharmacological testing, it has been shown that the certain N-aryl-piperazine derivatives bind to receptors of the 5-HT_{1A} type. Many of the N-aryl piperazine derivatives exhibit activity as 5-HT_{1A} antagonists. See, for example, U.S. Pat. No. 6,127,357, WO 97/03982, U.S. Pat. No. 6,469,007, and U.S. Pat. No. 6,586,436, the disclosures of which are incorporated herein by reference.

Certain N-aryl-piperazine derivatives are useful to treat a subject suffering from central nervous system (CNS) disorders such as schizophrenia, (and other psychotic disorders such as paranoia and mano-depressive illness), Parkinson's disease and other motor disorders, anxiety (e.g., generalized anxiety disorders, panic attacks, and obsessive compulsive disorders), depression (such as by the potentiation of serotonin reuptake inhibitors and serotonin norepinephrine reuptake inhibitors), Tourette's syndrome, migraine, autism, attention deficit disorders and hyperactivity disorders. These compounds may also be useful for the treatment of sleep disorders, social phobias, pain, thermoregulatory disorders, endocrine disorders, urinary incontinence, vasospasm, stroke, eating disorders such as for example obesity, anorexia and bulimia, sexual dysfunction, and the treatment of alcohol, drug and nicotine withdrawal. In addition, these compounds are useful for the treatment of cognitive dysfunction and may be useful for the treatment of cognitive dysfunction associated with mild cognitive impairment (MCI)) Alzheimer's disease and other dementias, including Lewy Body, vascular, and post stroke dementias. Cognitive dysfunction associated with surgical procedures, traumatic brain injury or stroke may also be treated with such compounds. Further, these compounds may be useful for the treatment of diseases in which cognitive dysfunction is a co-morbidity such as, for example, Parkinson's disease, autism, and attention deficit disorders.

A multi-step synthesis of certain N-aryl-piperazine derivatives of formula I: 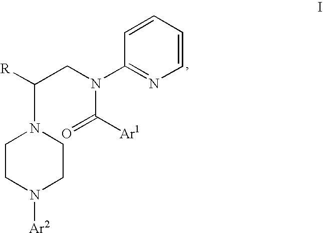
including (R)-4-cyano-N-[2-[4-(2,3-dihydro-1,4-benzodioxan-5-yl)-1-piperazinyl-propyl]-N-(2-pyridinyl)-benzamide is disclosed in U.S. Published Application No. 20030204087. The reaction scheme for (R)-4-cyano-N-[2-[4-(2,3-dihydro-1,4-benzodioxan-5-yl)-1-piperazinyl-propyl]-N-(2-pyridinyl)-benzamide is shown in Scheme 1, where benzodioxane aniline 2 is dialkylated with chloroethanol or ethyl bromoacetate. The diol intermediate 3 is either prepared directly or it is reduced from the corresponding diester 4. The diol moiety is activated as the dimesylate 5 followed by coupling with the aminosulfonic acid 6. Hydrolysis of the sulfonic acid moiety of compound 7 gives {2-[4-(2,3-Dihydro-benzo[1,4]dioxin-5-yl)-piperazin-1-yl]-propyl}-pyridin-2-yl-amine) 8. Acylation of compound 8 with p-cyanobenzoyl chloride gives the desired N-aryl-piperazine derivative, (R)-4-cyano-N-[2-[4-(2,3-dihydro-1,4-benzodioxan-5-yl)-1-piperazinyl-propyl]-N-(2-pyridinyl)-benzamide 1.

Although useful for laboratory preparations, the above conditions of the (R)-4-cyano-N-[2-[4-(2,3-dihydro-1,4-benzodioxan-5-yl)-1-piperazinyl-propyl]-N-(2-pyridinyl)-benzamide are less suitable for a commercial scale process. Disadvantages include the requirement for the purification of intermediates and the difficult isolation of the final product. Polyalkylation by-products form from alkylation of benzodioxane aniline 2 and by-products form during piperazine formation from dimesylate 5 and aminosulfonic acid 6 accumulate and make the crystallization of the piperazine salt difficult. This consequently affects the yield and purity of the final product.

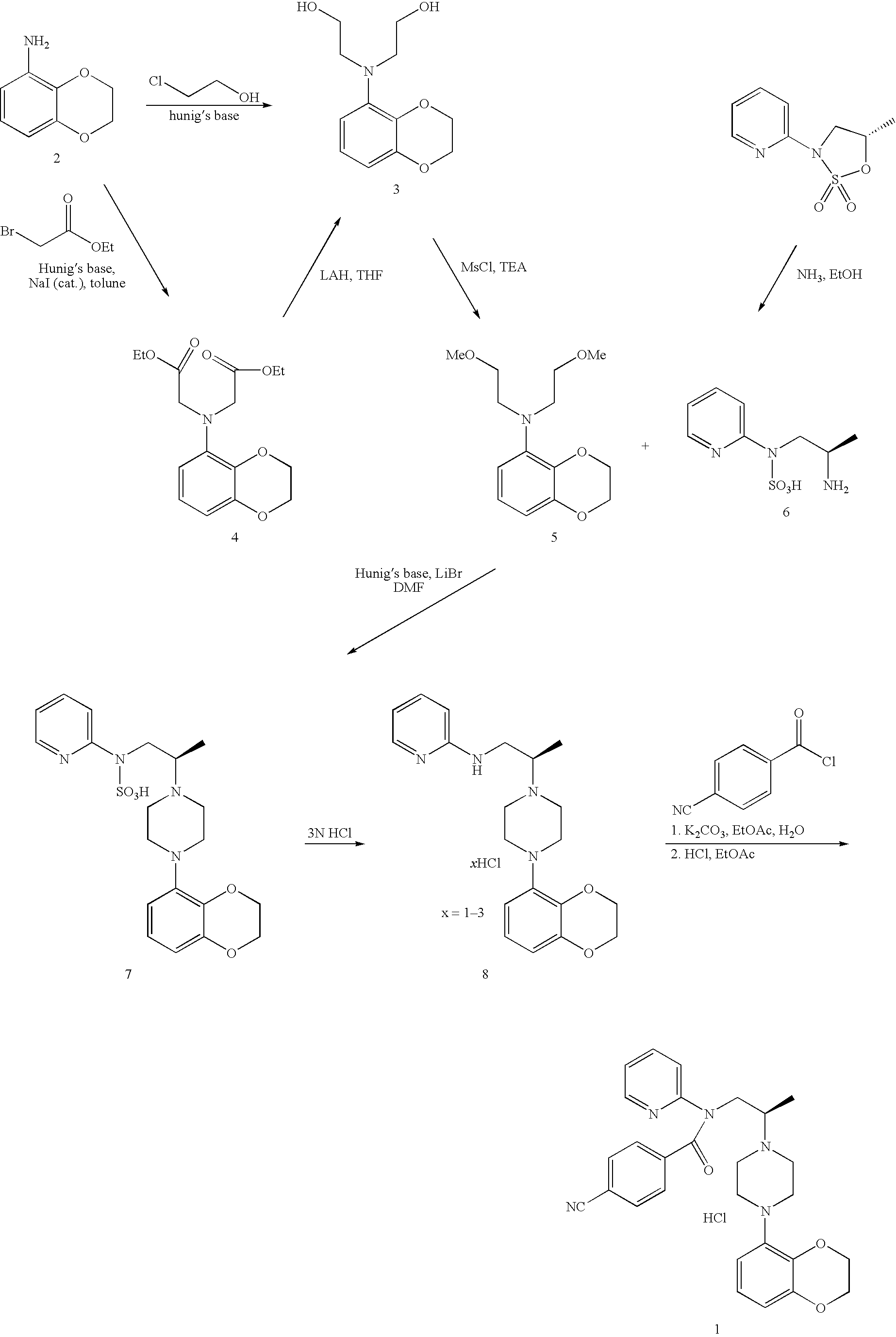

### SUMMARY OF THE INVENTION

The present invention is directed to processes for preparing N-aryl-piperazine derivatives, compositions comprising N-aryl-piperazine derivatives and a low level of common impurities, and the products produced by the improved processes.

In one embodiment, the present invention is directed to processes for preparing N-aryl-piperazine derivatives of formula I, comprising the steps of:

dialkylating an amino-substituted aryl compound or amino-substituted heteroaryl compound with 2-halooethanol to form a compound of formula II:

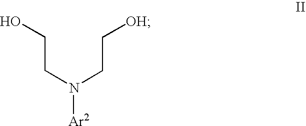

forming a salt of the compound of formula II;

activating the salt of the compound of formula II to form a compound of formula III:

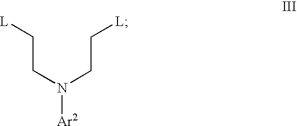

coupling the compound of formula III with aminoalkyl(pyridine-2-yl)sulfamic acid to form a compound of formula IV:

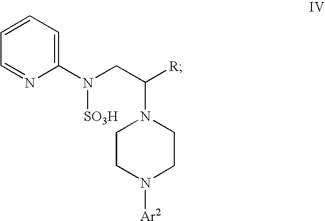

hydrolyzing the compound of formula IV to form a compound of formula V:

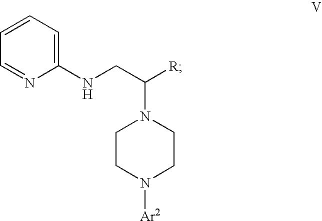

forming a salt of the compound of formula V;

acylating the salt of compound of formula V with a compound of formula VI:

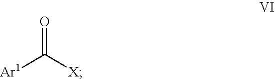

to form a product comprising a compound of formula I:

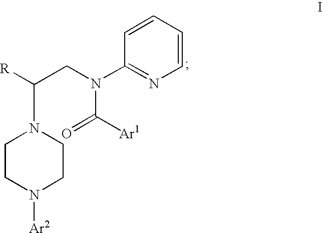

or pharmaceutically acceptable salt thereof;

wherein

R is C₁-C₄ alkyl;

Ar¹ is C₆-C₁₂ aryl optionally substituted with up to three substituents independently selected from the group consisting of halo, alkyl, alkoxy, alkoxycarbonyl, nitro, amino, alkylamino, dialkylamino, haloalkyl, dihaloalkyl, trihaloalkyl, cyano, and amido substituents each having no more than six carbon atoms;

Ar² is dihydrobenzodioxinyl, benzodioxinyl, or phenyl optionally substituted with up to three substituents independently selected from the group consisting of halo, methoxy, halomethyl, dihalomethyl, and trihalomethyl;

L is a leaving group; and

X is halo.

In another embodiment, the present invention is directed to the product produced by the process described above.

In yet another embodiment, the present invention is directed to compositions, comprising:

at least one compound of formula I:

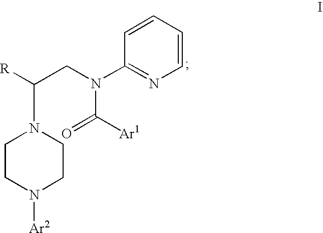

or pharmaceutically acceptable salt thereof;

wherein

R is C₁-C₄ alkyl;

Ar¹ is C₆-C₁₂ aryl optionally substituted with up to three substituents independently selected from the group consisting of halo, alkyl, alkoxy, alkoxycarbonyl, nitro, amino, alkylamino, dialkylamino, haloalkyl, dihaloalkyl, trihaloalkyl, cyano, and amido substituents each having no more than six carbon atoms;

Ar² is dihydrobenzodioxinyl, benzodioxinyl, or phenyl optionally substituted with up to three substituents independently selected from the group consisting of halo, methoxy, halomethyl, dihalomethyl, and trihalomethyl;

less than about 0.5% by weight, based on the weight of the composition, of a dimer of the compound of formula I; and

less than about 0.5% by weight, based on the weight of the composition, of a polyalkylation impurity.

### DETAILED DESCRIPTION OF THE INVENTION

The present invention is directed to processes for preparing N-aryl-piperazine derivatives, compositions comprising N-aryl-piperazine derivatives and low levels of common impurities, and products produced by the improved processes.

The following definitions are provided for the full understanding of terms and abbreviations used in this specification.

As used herein and in the appended claims, the singular forms “a,” “an,” and “the” include the plural reference unless the context clearly indicates otherwise. Thus, for example, a reference to “a compound” is a reference to one or more compounds and equivalents thereof known to those skilled in the art, and so forth.

The abbreviations in the specification correspond to units of measure, techniques, properties, or compounds as follows: “min” means minutes, “h” means hour(s), “μL” means microliter(s), “mL” means milliliter(s), “mM” means millimolar, “M” means molar, “mmole” means millimole(s), “cm” means centimeters, “SEM” means standard error of the mean and “IU” means International Units. “Δ° C.” and Δ “ED₅₀ value” means dose which results in 50% alleviation of the observed condition or effect (50% mean maximum endpoint).

In the context of this disclosure, a number of terms shall be utilized. The term “treatment” as used herein includes preventative (e.g., prophylactic), curative or palliative treatment and “treating” as used herein also includes preventative, curative and palliative treatment.

The terms “component”, “drug” or “pharmacologically active agent” or “active agent” or “medicament” are used interchangeably herein to refer to a compound or compounds or composition of matter which, when administered to an organism (human or animal) induces a desired pharmacologic and/or physiologic effect by local and/or systemic action.

The term “modulation” refers to the capacity to either enhance or inhibit a functional property of a biological activity or process, for example, receptor binding or signaling activity. Such enhancement or inhibition may be contingent on the occurrence of a specific event, such as activation of a signal transduction pathway and/or may be manifest only in particular cell types. The modulator is intended to comprise any compound, e.g., antibody, small molecule, peptide, oligopeptide, polypeptide, or protein, preferably small molecule, or peptide.

As used herein, “amino-substituted aryl compound” means a phenyl or naphthyl compound substituted with at least one amino substituent and optionally substituted with up to three substituents independently selected from the group consisting of halo, methoxy, halomethyl, dihalomethyl, and trihalomethyl. Such amino-substituted aryl compounds include phenylamine (also known as aniline) and naphthalenylamine.

As used herein, “amino-substituted heteroaryl compound” means a dihydrobenzodioxinyl or benzodioxinyl compound substituted with at least one amino substituent and optionally substituted with up to three substituents independently selected from the group consisting of halo, methoxy, halomethyl, dihalomethyl and trihalomethyl. Such amino-substituted heteroaryl compounds include benzodioxane aniline and dihydrobenzodioxane aniline.

Within the present invention, the compounds of formula I may be prepared in the form of pharmaceutically acceptable salts. As used herein, the term “pharmaceutically acceptable salts” refers to salts prepared from pharmaceutically acceptable non-toxic acids, including inorganic salts, and organic salts. Suitable non-organic salts include inorganic and organic acids such as acetic, benzenesulfonic, benzoic, camphorsulfonic, citric, ethenesulfonic, fumaric, gluconic, glutamic, hydrobromic, hydrochloric, isethionic, lactic, malic, maleic, mandelic, methanesulfonic, mucic, nitric, pamoic, pantothenic, phosphoric, succinic, sulfuric, tartaric acid, p-toluenesulfonic and the like. Particularly preferred are hydrochloric, hydrobromic, phosphoric, and sulfuric acids, and most preferably is the hydrochloride salt.

The term “subject” or “patient” refers to an animal including the human species that is treatable with the compositions, and/or methods of the present invention.

“Alkyl,” as used herein, refers to an aliphatic hydrocarbon chain and includes straight and branched chains such as methyl, ethyl, n-propyl, isopropyl, n-butyl, isobutyl, sec-butyl, t-butyl, n-pentyl, isopentyl, neo-pentyl, n-hexyl, and isohexyl. Lower alkyl refers to alkyl having 1 to 3 carbon atoms.

“Alkoxy,” as used herein, refers to the group R—O— where R is an alkyl group of 1 to 6 carbon atoms.

“Alkoxycarbonyl,” as used herein, refers to the group R—O—C(═O)— where R is an alkyl group of 1 to 6 carbon atoms.

“Halogen” (or “halo”), as used herein, refers to chlorine, bromine, fluorine, and iodine.

In one embodiment, the present invention is directed to processes, comprising the steps of:

dialkylating an amino-substituted aryl compound or amino-substituted heteroaryl compound with 2-halooethanol to form a compound of formula II:

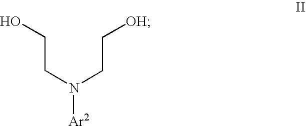

forming a salt of the compound of formula II;

activating the salt of the compound of formula II to form a compound of formula III:

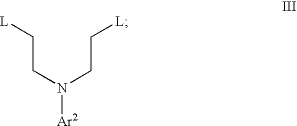

coupling the compound of formula III with aminoalkyl(pyridine-2-yl)sulfamic acid to form a compound of formula IV:

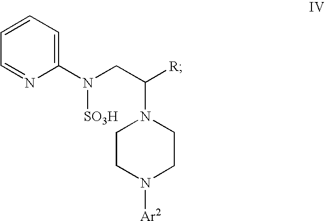

hydrolyzing the compound of formula IV to form a compound of formula V:

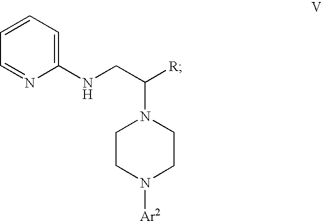

forming a salt of the compound of formula V;

acylating the salt of compound of formula V with a compound of formula VI:

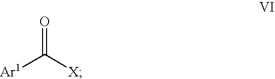

to form a product comprising a compound of formula I

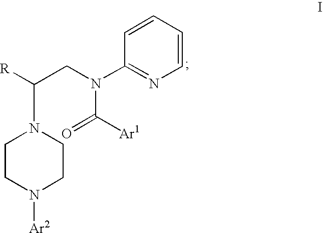

or pharmaceutically acceptable salt thereof;

wherein

R is C₁-C₄ alkyl;

Ar¹ is C₆-C₁₂ aryl optionally substituted with up to three substituents independently selected from the group consisting of halo, alkyl, alkoxy, alkoxycarbonyl, nitro, amino, alkylamino, dialkylamino, haloalkyl, dihaloalkyl, trihaloalkyl, cyano, and amido substituents each having no more than six carbon atoms;

Ar² is dihydrobenzodioxinyl, benzodioxinyl, or phenyl optionally substituted with up to three substituents independently selected from the group consisting of halo, methoxy, halomethyl, dihalomethyl, and trihalomethyl;

L is a leaving group; and

X is halo.

In preferred embodiments, the aminoalkyl(pyridine-2-yl)sulfamic acid is 2-aminopropyl(pyridine-2-yl)sulfamic acid, more preferably, (2R)-2-aminopropyl(pyridine-2-yl)sulfamic acid. In certain preferred embodiments, the aminoalkyl(pyridine-2-yl)sulfamic acid is formed from its 2-(5-alkyl-2,2-dioxo-2λ⁶-[1,2,3]oxathiazolidin-3-yl)-pyridine precursor, preferably, 2-(5-methyl-2,2-dioxo-2λ⁶-[1,2,3]oxathiazolidin-3-yl)-pyridine precursor.

In certain preferred embodiments, R is methyl.

In certain preferred embodiments, Ar¹ is a substituted phenyl, preferably, a cyano substituted phenyl, more preferably, p-cyanophenyl.

In certain preferred embodiments, Ar² is dihydrobenzodioxinyl.

In certain preferred embodiments, L is halo, tosylate, mesylate, or p-bromophenylsulfonyloxy, preferably, mesylate or halo (especially chloro or bromo), most preferably, mesylate.

In certain preferred embodiments, X is chloro.

In certain preferred embodiments, the amino-substituted aryl compound is aniline or naphthalenylamine. In other preferred embodiments, the amino-substituted heteroaryl compound is benzodioxane aniline or dihydrobenzodioxane aniline, preferably dihydrobenzodioxane aniline.

In certain preferred embodiments, the tertiary amine is triethylamine.

In certain preferred embodiments, the 2-halooethanol is 2-chloroethanol.

In certain preferred embodiments, the dialkylating step is carried out in the presence of at least one inorganic base in aprotic solvent.

In certain preferred embodiments, the salt of the compound of formula II is an inorganic salt, preferably a hydrogen chloride salt.

In certain preferred embodiments, the salt of the compound of formula V is an inorganic salt, preferably a hydrogen chloride salt.

In certain preferred embodiments, the protecting step or the activating step is carried at a temperature of from about 0° C. to about 25° C., preferably, at a temperature of from about 0° C. to about 15° C., more preferably, at a temperature of from about 0° C. to about 10° C.

The process of the invention is particularly useful for the preparation of the compounds of formula I of formula IA:

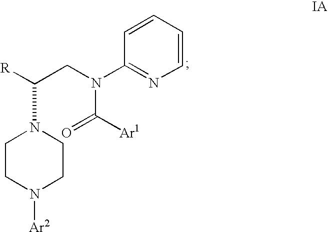

or pharmaceutically acceptable salt thereof.

The process of the invention is particularly useful for the preparation of 4-cyano-N-{2-[4-(2,3-dihydro-benzo[1,4]dioxin-5-yl)-piperazin-1-yl]-propyl}-N-pyridin-2-yl-benzamide or pharmaceutically acceptable salt thereof, including especially, (R)-4-cyano-N-[2-[4-(2,3-dihydro-1,4-benzodioxan-5-yl)-1-piperazinyl-propyl]-N-(2-pyridinyl)-benzamide or pharmaceutically acceptable salt thereof.

The improved process of the invention is characterized by improved product purity, higher yields, lower costs, and technical convenience relative to synthetic route disclosed in U.S. Published Application No. 20030204087. For example, with respect to the formation of 4-cyano-N-{2-[4-(2,3-dihydro-benzo[1,4]dioxin-5-yl)-piperazin-1-yl]-propyl}-N-pyridin-2-yl-benzamide, dialkylation of benzodioxane anline with 2-chloroethanol is catalyzed with inorganic bases in aprotic solvent. Polyalkylation is minimized under these conditions. Furthermore, simple filtration of the inorganic salt formed during the reaction avoids the need for multiple extractions of the highly water-soluble diol. Subsequent salt formation of diol gives a stable solid that can be readily isolated in high yield and purity.

Yield and rate of aminosulfonic acid formation from ammonia opening of sulfamate is improved with a higher concentration and molar equivalent of ammonia used. A highly pure and crystalline product was isolated and purified simply from solvent exchange with acetonitrile. The solid aminosulfonic acid product 6 is readily isolated by filtration.

The diol salt may be telescoped to the piperazine salt in high yield and purity. Activation of the diol can be accomplished using methanesulfonic (mesyl) anhydride or methanesulfonyl (mesyl) chloride, preferably methanesulfonyl chloride. Dimesylate is formed directly from the diol salt. The reaction may be carried out at low temperatures, for example, in the range of about 0° C. to about 25° C., preferably, about 0° C. to about 25° C., more preferably about 0° C. to about 10° C. to prevent chloride displacement of the mesylate. The dimesylate formed is then added to a warm mixture of aminosulfonic acid and Hünig's base in butyronitrile to give piperazine sulfonic acid. The sulfonic acid moiety of piperazine is then hydrolyzed using a 3 N HCl solution. The piperazine is purified and isolated by filtration as the piperazine salt. The salts formed allow for easy isolation and purification of the piperazine.

The dialkylation of benzodioxane aniline with chloroethanol is preferably carried out in an aprotic solvent, such as xylene, toluene, propionitrile, and butyronitrile, preferably high boiling polar, aprotic solvent such as propionitrile, and butyronitrile, and most preferably butyronitrile, at a temperature of from about 45° C. to about 120° C., preferably at about 50° C., to reflux in the presence of a base, preferably in an inorganic bases such as potassium carbonate or sodium carbonate, catalyzed by catalysts such as sodium iodide or t-butylammonium iodide. The use of inorganic base gives an inorganic salt by-product that may be easily removed by filtration. The diol product is highly water-soluble and the use of an inorganic base avoids the need for an aqueous work-up. A salt, preferably the hydrochloride of the aniline diol, can be obtained for easy isolation in high yield and purity.

Yield and conversion rate of the ammonia opening of sulfamate was improved with increased molar equivalent of ammonia used. This step is characterized by easy isolation of product from solvent exchange with CH₃CN.

Piperazine formation step is carried out from coupling of the activated diol and aminosulfonic acid. Importantly, the piperazine may be prepared from the diol salt and aminosulfonic acid without isolation of intermediates, without changing the solvent (preferably butyronitrile), and with high yield and purity.

Furthermore, the modifications of the present process are less labor- and time-intensive than the synthetic route disclosed in U.S. Published Application No. 20030204087.

In other embodiments, the invention is directed to the products produced by the improved process that contain lower levels of common impurities, including the dimer of the compound of formula I and the polyalkylation products. The dimer impurities of the compound of formula I include the compounds of formula A:

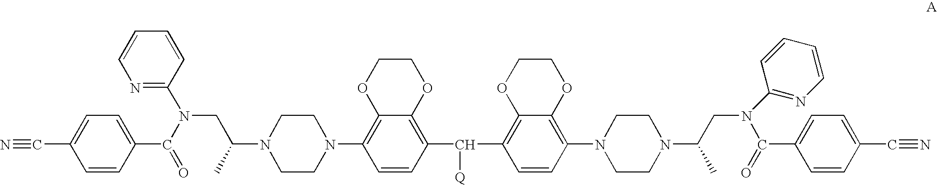

wherein Q is H, —CH₃, —CH(CH₃)₂, CH(CH₂)₂CH₃, or CH(CH₂)₃CH₃.

In yet other embodiments, the invention is directed to compositions, comprising:

the product produced by the improved process; and

at least one pharmaceutically acceptable carrier.

In another embodiment, the invention is directed to compositions, comprising:

at least one compound of formula I:

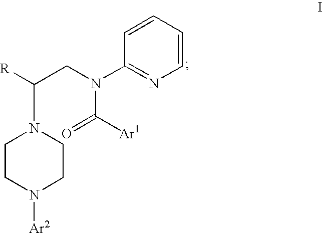

or pharmaceutically acceptable salt thereof;

wherein

R is C₁-C₄ alkyl;

Ar¹ is C₆-C₁₂ aryl optionally substituted with up to three substituents independently selected from the group consisting of halo, alkyl, alkoxy, alkoxycarbonyl, nitro, amino, alkylamino, dialkylamino, haloalkyl, dihaloalkyl, trihaloalkyl, cyano, and amido substituents each having no more than six carbon atoms;

Ar² is dihydrobenzodioxinyl, benzodioxinyl, or phenyl optionally substituted with up to three substituents independently selected from the group consisting of halo, methoxy, halomethyl, dihalomethyl, and trihalomethyl;

less than about 0.5% by weight, based on the weight of the composition, of a dimer of the compound of formula I; and

less than about 0.5% by weight, based on the weight of the composition, of a polyalkylation impurity.

In certain preferred embodiments, the composition further comprises at least one pharmaceutically acceptable carrier.

In certain preferred embodiments, R is methyl.

In certain preferred embodiments, Ar¹ is a substituted phenyl, preferably, cyano substituted phenyl, more preferably, p-cyanophenyl.

In certain preferred embodiments, Ar² is dihydrobenzodioxinyl.

In certain preferred embodiments, L is a mesylate or halo, especially chloro or bromo, preferably, mesylate.

In certain preferred embodiments, the compound of formula I is a compound of formula IA:

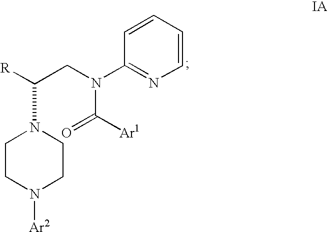

or a pharmaceutical acceptable salt thereof.

In certain preferred embodiments, the compound of formula I is 4-cyano-N-{2-[4-(2,3-dihydro-benzo[1,4]dioxin-5-yl)-piperazin-1 -yl]-propyl}-N-pyridin-2-yl-benzamide or pharmaceutically acceptable salt thereof, preferably (R)-4-cyano-N-[2-[4-(2,3-dihydro-1,4-benzodioxan-5-yl)-1-piperazinyl-propyl]-N-(2-pyridinyl)-benzamide or pharmaceutically acceptable salt thereof.

Some of the compounds of the present invention may contain chiral centers and such compounds may exist in the form of stereoisomers (i.e. enantiomers). The present invention includes all such stereoisomers and any mixtures thereof including racemic mixtures. Racemic mixtures of the stereoisomers as well as the substantially pure stereoisomers are within the scope of the invention. The term “substantially pure,” as used herein, refers to at least about 90 mole %, more preferably at least about 95 mole %, and most preferably at least about 98 mole % of the desired stereoisomer is present relative to other possible stereoisomers. Preferred enantiomers may be isolated from racemic mixtures by any method known to those skilled in the art, including high performance liquid chromatography (HPLC) and the formation and crystallization of chiral salts or prepared by methods described herein. See, for example, Jacques, et al., Enantiomers, Racemates and Resolutions (Wiley lnterscience, New York, 1981); Wilen, S. H., et al., Tetrahedron, 33:2725 (1977); Eliel, E. L. Stereochemistry of Carbon Compounds, (McGraw-Hill, NY, 1962); Wilen, S. H. Tables of Resolving Agents and Optical Resolutions, p. 268 (E. L. Eliel, Ed., University of Notre Dame Press, Notre Dame, Ind. 1972).

The present invention includes prodrugs of the compounds of formula I. “Prodrug,” as used herein, means a compound which is convertible in vivo by metabolic means (e.g. by hydrolysis) to a compound of formula I. Various forms of prodrugs are known in the art, for example, as discussed in Bundgaard, (ed.), Design of Prodrugs, Elsevier (1985); Widder, et al. (ed.), Methods in Enzymology, vol. 4, Academic Press (1985); Krogsgaard-Larsen, et al., (ed). “Design and Application of Prodrugs,” Textbook of Drug Design and Development, Chapter 5, 113-191 (1991), Bundgaard, et al., Journal of Drug Deliver Reviews, 1992, 8:1-38, Bundgaard, J. of Pharmaceutical Sciences, 1988, 77:285 et seq.; and Higuchi and Stella (eds.) Prodrugs as Novel Drug Delivery Systems, American Chemical Society (1975).

Further, the compounds of formula I may exist in unsolvated as well as in solvated forms with pharmaceutically acceptable solvents such as water, ethanol, and the like. In general, the solvated forms are considered equivalent to the unsolvated forms for the purpose of the present invention.

The compounds can be synthesized, for example, by the methods described below, or variations thereon as appreciated by the skilled artisan. All processes disclosed in association with the present invention are contemplated to be practiced on any scale, including milligram, gram, multigram, kilogram, multikilogram or commercial industrial scale.

As will be readily understood, functional groups present may contain protecting groups during the course of synthesis. Protecting groups are known per se as chemical functional groups that can be selectively appended to and removed from functionalities, such as hydroxyl groups and carboxyl groups. These groups are present in a chemical compound to render such functionality inert to chemical reaction conditions to which the compound is exposed. Any of a variety of protecting groups may be employed with the present invention. Protecting groups that may be employed in accordance with the present invention may be described in Greene, T. W. and Wuts, P. G. M., Protective Groups in Organic Synthesis 2d. Ed., Wiley & Sons, 1991.

Compounds of the present invention are suitably prepared in accordance with the following general description and specific examples. Variables used are as defined for Formula I, unless otherwise noted. The reagents used in the preparation of the compounds of this invention can be either commercially obtained or can be prepared by standard procedures described in the literature.

The process described above may be carried out to give a compound of formula I in the form of a free base or as an acid addition salt. If the compound of formula I is obtained as an acid addition salt, the free base can be obtained by basifying a solution of the acid addition salt. Conversely, if the product of the process is a free base an acid addition salt, particularly a pharmaceutically acceptable acid addition salt, may be obtained by dissolving the free base in a suitable organic solvent and treating the solution with an acid, in accordance with conventional procedures for preparing acid addition salts from base compounds.

Examples of acid addition salts are those formed from inorganic and organic acids, such as sulfuric, hydrochloric, hydrobromic, phosphoric, tartaric, fumaric, maleic, citric, acetic, formic, methanesulfonic, p-toluenesulfonic, oxalic, and succinic acids.

The compounds of formula I may contain one or more asymmetric carbon atoms, so that some compounds can exist in different stereoisomeric forms. The compounds can be, for example, racemates or optically active forms. The optically active forms can be obtained by resolution of the racemates or by asymmetric synthesis.

The compounds of formula I possess pharmacological activity. In particular, they act on the central nervous system (CNS) by binding to 5-HT receptors. In pharmacological testing, it has been shown that the compounds particularly bind to receptors of the 5-HT_{1A} type. In general, the compounds selectively bind to receptors of the 5-HT_{1A} type to a much greater extent than they bind to other receptors, such as α₁ and D₂ receptors. Many exhibit activity as 5-HT_{1A} antagonists in pharmacological testing.

The compositions and products produced by the process of the present invention comprising compounds of formula I are useful to treat a subject suffering from central nervous system (CNS) disorders such as schizophrenia, (and other psychotic disorders such as paranoia and mano-depressive illness), Parkinson's disease and other motor disorders, anxiety (e.g., generalized anxiety disorders, panic attacks, and obsessive compulsive disorders), depression (such as by the potentiation of serotonin reuptake inhibitors and serotonin norepinephrine reuptake inhibitors), Tourette's syndrome, migraine, autism, attention deficit disorders and hyperactivity disorders. These compositions and products produced by the process of the present invention may also be useful for the treatment of sleep disorders, social phobias, pain, thermoregulatory disorders, endocrine disorders, urinary incontinence, vasospasm, stroke, eating disorders such as for example obesity, anorexia and bulimia, sexual dysfunction, and the treatment of alcohol, drug and nicotine withdrawal. In addition, these compositions and products produced by the process of the present invention are useful for the treatment of cognitive dysfunction and may be useful for the treatment of cognitive dysfunction associated with mild cognitive impairment (MCI)) Alzheimer's disease and other dementias, including Lewy Body, vascular, and post stroke dementias. Cognitive dysfunction associated with surgical procedures, traumatic brain injury or stroke may also be treated with the compositions and products produced by the process of the present invention. Further, these compositions and products produced by the process of the present invention may be useful for the treatment of diseases in which cognitive dysfunction is a co-morbidity such as, for example, Parkinson's disease, autism, and attention deficit disorders.

In other embodiments, the invention is directed to compositions, comprising:

a. at least compound of formula I or pharmaceutically acceptable salt thereof;

b. less than about 0.5% by weight, based on the weight of the composition, of a dimer of the compound of formula I;

c. less than about 0.5% by weight, based on the weight of the composition, of a polyalkylation impurity; and

d. optionally, at least one pharmaceutically acceptable carrier.

Generally, the compound of formula I or a pharmaceutically acceptable salt thereof will be present at a level of from about 0.1%, by weight, to about 90% by weight, based on the total weight of the composition, based on the total weight of the composition. Preferably, the compound of formula I or a pharmaceutically acceptable salt thereof will be present at a level of at least about 1% by weight, based on the total weight of the composition. More preferably, the compound of formula I or a pharmaceutically acceptable salt thereof will be present at a level of at least about 5% by weight, based on the total weight of the composition. Even more preferably, the compound of formula I or a pharmaceutically acceptable salt thereof will be present at a level of at least about 10% by weight, based on the total weight of the composition. Yet even more preferably, the compound of formula I or a pharmaceutically acceptable salt thereof will be present at a level of at least about 25% by weight, based on the total weight of the composition.

Preferably, the compositions of the invention comprise less than about 0.2% by weight of the dimer of the compound of formula I, based on the total weight of the composition. More preferably, the compositions of the invention comprise less than about 0.2% by weight of the dimer of the compound of formula I, based on the total weight of the composition. Even more preferably, the compositions of the invention is substantially free of the dimer of the compound of formula I.

Preferably, the compositions of the invention comprise less than about 0.2% by weight of the polyalkylation impurity, based on the total weight of the composition. More preferably, the compositions of the invention comprise less than about 0.1% by weight of the polyalkylation impurity, based on the total weight of the composition. Even more preferably, the compositions of the invention is substantially free of the polyalkylation impurity.

Such compositions are prepared in accordance with acceptable pharmaceutical procedures, such as described in Remington's Pharmaceutical Sciences, 17th edition, ed. Alfonoso R. Gennaro, Mack Publishing Company, Easton, Pa. (1985). Pharmaceutically acceptable carriers are those that are compatible with the other ingredients in the formulation and biologically acceptable.

The compounds and compositions of this invention may be administered orally or parenterally, neat or in combination with conventional pharmaceutical carriers. Applicable solid carriers can include one or more substances that may also act as flavoring agents, lubricants, solubilizers, suspending agents, fillers, glidants, compression aids, binders or tablet-disintegrating agents or an encapsulating material. In powders, the carrier is a finely divided solid that is in admixture with the finely divided active ingredient. In tablets, the active ingredient is mixed with a carrier having the necessary compression properties in suitable proportions and compacted in the shape and size desired. The powders and tablets preferably contain up to 99% of the active ingredient. Suitable solid carriers include, for example, calcium phosphate, magnesium stearate, talc, sugars, lactose, dextrin, starch, gelatin, cellulose, methyl cellulose, sodium carboxymethyl cellulose, polyvinylpyrrolidine, low melting waxes and ion exchange resins.

Liquid carriers may be used in preparing solutions, suspensions, emulsions, syrups, and elixirs. The active ingredient of this invention can be dissolved or suspended in a pharmaceutically acceptable liquid carrier such as water, an organic solvent, a mixture of both or pharmaceutically acceptable oils or fat. The liquid carrier can contain other suitable pharmaceutical additives such as solubilizers, emulsifiers, buffers, preservatives, sweeteners, flavoring agents, suspending agents, thickening agents, colors, viscosity regulators, stabilizers, or osmo-regulators. Suitable examples of liquid carriers for oral and parenteral administration include water (particularly containing additives as above, e.g. cellulose derivatives, preferably sodium carboxymethyl cellulose solution), alcohols (including monohydric alcohols and polyhydric alcohols e.g. glycols) and their derivatives, and oils (e.g. fractionated coconut oil and arachis oil). For parenteral administration, the carrier can also be an oily ester such as ethyl oleate and isopropyl myristate. Sterile liquid carriers are used in sterile liquid form compositions for parenteral administration.

Liquid pharmaceutical compositions, which are sterile solutions or suspensions, can be administered by, for example, intramuscular, intraperitoneal or subcutaneous injection. Sterile solutions can also be administered intravenously. Oral administration may be either liquid or solid composition form.

Preferably the pharmaceutical composition is in unit dosage form, e.g. as tablets, capsules, powders, solutions, suspensions, emulsions, granules, or suppositories. In such form, the composition is sub-divided in unit dose containing appropriate quantities of the active ingredient; the unit dosage forms can be packaged compositions, for example packeted powders, vials, ampoules, prefilled syringes or sachets containing liquids. The unit dosage form can be, for example, a capsule or tablet itself, or it can be the appropriate number of any such compositions in package form.

In another embodiment of the present invention, the compositions and products produced by the process of the present invention may be administered to a mammal with one or more other pharmaceutical active agents, such as those agents being used to treat any other medical condition present in the mammal. Examples of such pharmaceutical active agents include pain relieving agents, anti-angiogenic agents, anti-neoplastic agents, anti-diabetic agents, anti-infective agents, or gastrointestinal agents, or combinations thereof.

The one or more other pharmaceutical active agents may be administered in a therapeutically effective amount simultaneously (such as individually at the same time, or together in a pharmaceutical composition), and/or successively with one or more compounds of the present invention.

The term “combination therapy” refers to the administration of two or more therapeutic agents or compounds to treat a therapeutic condition or disorder described in the present disclosure, for example depression, anxiety, hypotension, sleeping disorders, eating disorders, sexual dysfunction, or other condition or disorder. Such administration includes use of each type of therapeutic agent in a concurrent manner. In either case, the treatment regimen will provide beneficial effects of the drug combination in treating the conditions or disorders described herein.

The route of administration may be any route, which effectively transports the active compound of formula I to the appropriate or desired site of action, such as oral, nasal, pulmonary, transdermal, such as passive or iontophoretic delivery, or parenteral, e.g. rectal, depot, subcutaneous, intravenous, intraurethral, intramuscular, intranasal, ophthalmic solution or an ointment. Furthermore, the administration of compound of formula I with other active ingredients may be concurrent or simultaneous.

### EXAMPLES

The present invention is further defined in the following Examples, in which all parts and percentages are by weight and degrees are Celsius, unless otherwise stated. It should be understood that these examples, while indicating preferred embodiments of the invention, are given by way of illustration only. From the above discussion and these examples, one skilled in the art can ascertain the essential characteristics of this invention, and without departing from the spirit and scope thereof, can make various changes and modifications of the invention to adapt it to various usages and conditions. The reagents and intermediates used herein are either commercially available or prepared according to standard literature procedures.

The synthesis of (R)-4-cyano-N-[2-[4-(2,3-dihydro-1,4-benzodioxan-5-yl)-1-piperazinyl-propyl]-N-(2-pyridinyl)-benzamide is shown in Scheme 2, as described in more detail below.

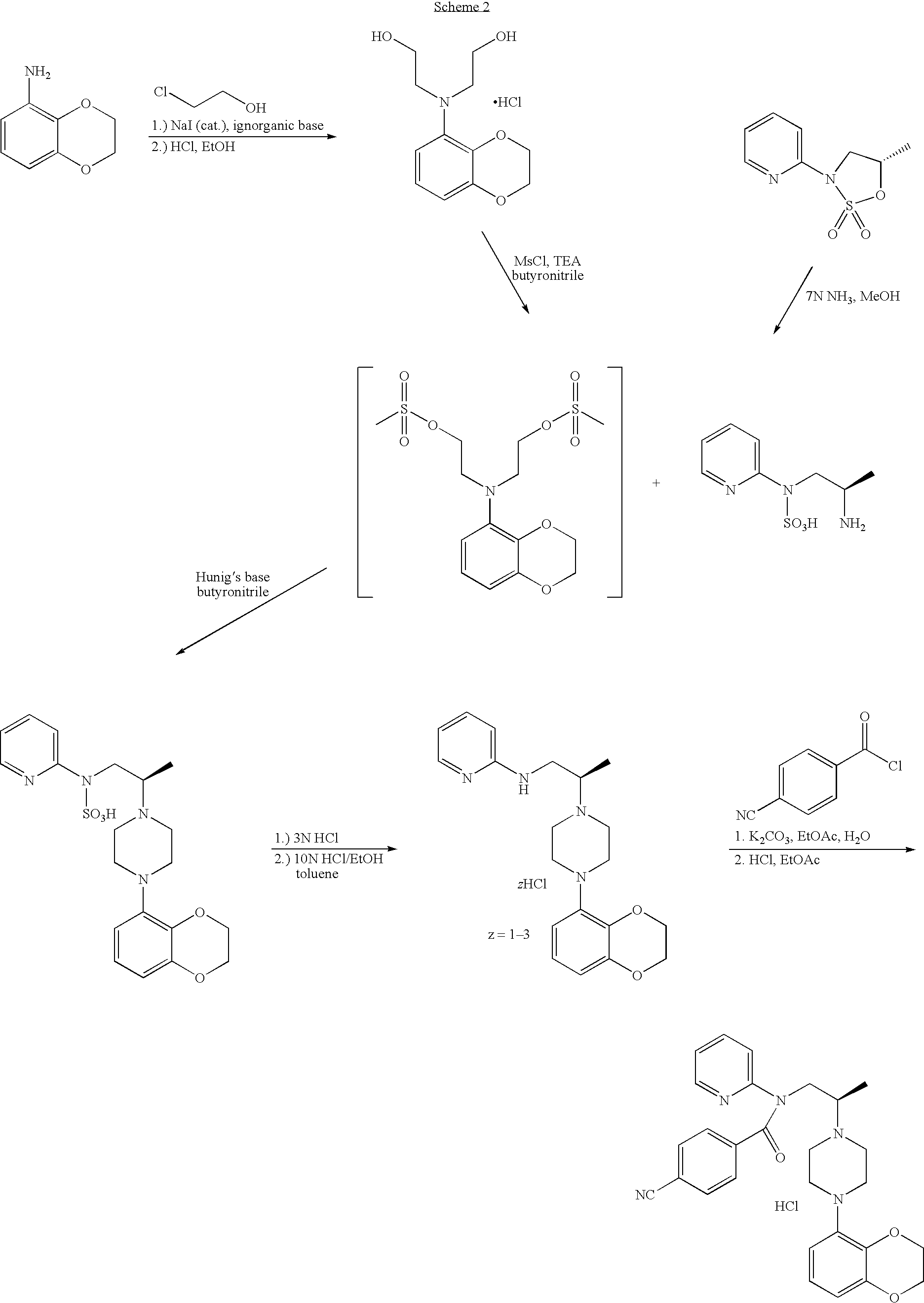

### Example 1

### Preparation of the Hydrogen Chloride Salt of 2-[(2,3-dihydro-benzo[1,4]dioxon-5-yl)-(2-hydroxy-ethyl)-amino]-ethanol

A solution of benzodioxane aniline (50 g, 0.331 mol) in butyronitrile (150 mL) at room temperature was added sodium carbonate (70.2 g, 0.662 mole), sodium iodide (5 g, 0.033 mole), and chloroethanol (160 g, 133 mL). The reaction mixture was heated to reflux (115±5° C.). After 16 hours, it was cooled to room temperature then the suspension was filtered through a Büchner funnel. The solids were washed with ethyl acetate (2×100 mL). The combined filtrates were concentrated to ¼ of its original volume. Toluene (150 mL) was added followed by the addition of 10 N HCl in EtOH (66 mL). The reaction mixture was stirred at 23-25° C. overnight for 16 hours and then cooled to 0-5° C. for 2 hours. The product was filtered through a Büchner funnel, washed with toluene (2×50 mL), dried in a vacuum oven to give 79 g (86%) of diol HCl as a light purple solid.

¹H NMR (CDCl₃) δ 6.83-6.70 (m, 3H), 4.29-4.20 (m, 4H), 3.54 (t, J=5.1 Hz, 4H), 3.20 (t, J=5.1 Hz, 4H), 3.09 (br s, 2H) (free base). mp: 145-148° C.

Potassium carbonate has been substituted for the sodium carbonate and t-butylammonium iodide has been substituted for the sodium iodide.

### Example 2

### Preparation of (2R)-2-aminopropyl(pyridin-2-yl)sulfamic acid

A mixture of sulfamate (25 g, 0.12 mol) in a 7.8 N ammonia in MeOH solution (270 mL, 2.1 mol) was stirred at room temperature under N₂ for one day. The resulting mixture was concentrated to ⅓ its original volume then acetonitrile (80 mL) was added. The mixture was concentrated to ⅓ its original volume. Another portion of acetonitrile (80 mL) was added and the mixture was again concentrated to ⅓ its original volume. The suspension was heated at 40-45° C. for 10-15 minutes and then cooled to room temperature. The off-white solids was then filtered, washed with acetonitrile (50 mL), and dried in a vacuum oven to give 22 g (81%) of the sulfamic acid as an off-white solid (98 area % by LC/MS).

¹H NMR (DMSO) δ 8.17 (d, J=3 Hz, 1H), 7.5-7.9 (m, 5H), 6.82 (t, J=4.5 Hz, 1H), 4.03 (dd, J=10.8 Hz, 3.6 Hz, 1H), 3.94 (dd, J=10.8 Hz, 5.7 Hz, 1H), 3.4-3.6 (m, 1H) 1.18 (d, J=5.1 Hz, 3H);

¹³C NMR (DMSO) δ 156.1, 146.8, 136.9, 115.7, 114.6, 50.1, 47.9, 16.7; IR (KBr): υₘₐₓ 3426, 3137, 3073, 2980, 2518, 1629, 1588, 1520, 1465, 1432, 1366, 1286, 1234, 1197, 1146, 1117, 1063, 1042 cm⁻¹; CHN (calculated) C, 41.6; H, 5.62; N, 18.2, CHN (observed) C, 41.1; H, 5.49; N, 17.7; m.p. 175.5-179° C.

### Example 3

### Preparation of the Hydrogen Chloride Salt of {2-[4-(2,3-dihydrobenzo-[1,4]dioxin-5-yl)-piperazin-1-yl]-propyl}-pyridin-2-yl-amine)

A mixture of diol•HCl (50.0 g. 0.18 mol) in butyronitrile (250 mL) was cooled to 8-9° C. in an ice-water bath. To this mixture, methanesulfonyl chloride (74 g, 0.64 mol) was added followed by addition of triethylamine (TEA) (150 mL, 1.07 mol) over 30 minutes while keeping reaction temperature less than about 25° C. After addition, the reaction mixture was allowed to stir in the ice-water bath for an additional 30 minutes. At the end of this time, cold water (300 mL) was added. The two layers were separated. The aqueous layer was extracted with butyronitrile (100 mL). The combined butyronitrile layers was extracted with saturated NaHCO₃ (300 mL) and H₂O (300 mL). This dimesylate in butyronitrile solution was then added over 30 minutes to a mixture of aminosulfonic acid (37.4 g, 0.16 mol) and Hünig's base (127 mL, 0.727 mol) in butyronitrile (150 mL) heated at 60-65° C. The mixture was heated for 3 hours. During this time, the mixture turned clear. The mixture was cooled to room temperature then 3 N HCl (250 mL) was added. This was stirred at room temperature for 2 hours then the two layers were separated. The aqueous layer was extracted with CH₂Cl₂ (2×250 mL) then cooled to 4-6° C. before ethyl acetate (EtOAc) (400 mL) was added followed by slow addition of 10 N NaOH (100 mL). (Note: pH of the aqueous layer was ˜10-11) The two layers were separated. The aqueous layer was extracted with EtOAc (150 mL). The combined EtOAc layers was extracted with H₂O (300 mL) then solvent exchanged with toluene (400 mL). To the toluene solution, ethyl alcohol (2B) (55 mL) was added. This was heated to 60-65° C. before 9.54 N HCl in ethanol (68 mL) was added slowly. The mixture cooled to room temperature and a solid formed. The mixture was stirred at room temperature overnight then cooled to 0-5° C. for 2-3 hours before filtration, obtained 32.6 g, 43% yield of the title compound as an off-white solid.

R_{f}=0.28 (20:1 CHCl₃:CH₃OH) (free base)

¹H NMR (CDCl₃) δ 8.37 (m, 1H), 7.30-7.65 (m, 2H), 6.77 (t, J=8.1 Hz, 1H), 6.40-6.60 (m, 1H), 6.40 (d, J=8.3 Hz, 1H), 5.37 (m, 1H), 4.15-4.40 (m, 4H), 3.30 (m, 1H), 2.90-3.20 (m, 4H), 2.82 (m, 1H), 2.62 (m, 1H), 1.10 (d, J=6.4 Hz, 3H).

### Example 4

### Preparation of the Hydrogen Chloride Salt of (R)-4-cyano-N-[2-[4-(2,3-dihydro-1,4-benzodioxin-5-yl)-1-piperazinyl-propyl]-N-(2-pyridinyl)-benzamide

To a solution of potassium carbonate (125.1 g, 906 mmol) in H₂O (150 mL), EtOAc (500 mL) was added. To the two phase mixture, hydrogen chloride salt of {2-[4-(2,3-dihydro-benzo[1,4]dioxin-5-yl)-piperazin-1-yl]-propyl}-pyridin-2-yl-amine (100 g, 216 mmol) in H₂O (300 mL) was added. The mixture was stirred and cooled to 0-5° C. before a solution of 4-cyanobenzoyl chloride (42.8 g, 259 mmol) in EtOAc (200 mL) was added over 30 minutes. The reaction mixture was warmed to 23-25° C. and stirred for an additional 1 hour. H₂O (300 mL) was added and stirred the mixture for 5 minutes. The two layers were separated. The aqueous layer was extracted with EtOAc (150 mL). The combined organic layers were washed with brine (300 mL) and H₂O (300 mL) then concentrated to ˜⅓ its original volume. Additional EtOAc (˜300 mL) was added to the reaction mixture. The solution was warmed to 63-67° C. HCl in EtOH (4.8 N, 50 mL) was added dropwise over 1 hour. Stirred at 63-67° C. for 10 minutes then cooled to 23-25° C. Off-white solids formed upon addition. After 1 hour of stirring at room temperature, the reaction mixture was cooled in a 0-5° C. ice-bath and stirred for an additional 1 hour. The solids were filtered, washed with EtOAc (2×100 mL) and dried under vacuum to give 90.4 g (80%) of the titled compound (98.6% by HPLC) as an off-white solid.

¹H NMR (DMSO) δ 8.45 (m, 1H), 7.69-7.78 (m, 3H), 7.45 (d, J=8.3 Hz, 2H), 7.28 (m, 1H), 7.20 (d, J=8.0 Hz, 1H), 6.78 (t, J=8.1 Hz, 1H), 6.58 (m, 2H), 4.41 (m, 2H), 4.25 (m, 4H), 3.50-3.90 (m, 8H), 3.39 (m, 1H), 3.19 (m, 1H), 1.40 (d, J=6.7 Hz, 3H).

The levels of dimer impurity and polyalkylation impurity were measured by high performance liquid chromatography (HPLC) with a detection limit of 0.05%, by weight, based on the weight of the total composition. For Example 4, the % HPLC showed no dimer impurity or polyalkylation impurity.

When ranges are used herein for physical properties, such as molecular weight, or chemical properties, such as chemical formulae, all combinations and subcombinations of ranges specific embodiments therein are intended to be included.

The disclosures of each patent, patent application and publication cited or described in this document are hereby incorporated herein by reference, in its entirety.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

1. A process, comprising the steps of:
dialkylating an amino-substituted aryl compound or amino-substituted heteroaryl compound with 2-halooethanol to form a compound of formula II: 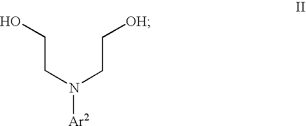
forming a salt of the compound of formula II;
activating the salt of the compound of formula II to form a compound of formula III: 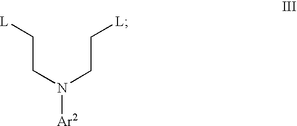
coupling the compound of formula III with aminoalkyl(pyridine-2-yl)sulfamic acid to form a compound of formula IV: 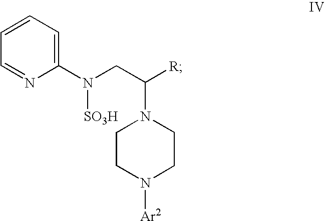
hydrolyzing the compound of formula IV to form a compound of formula V: 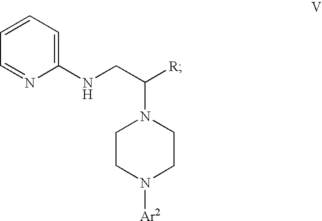
forming a salt of the compound of formula V;
acylating the salt of compound of formula V with a compound of formula VI: 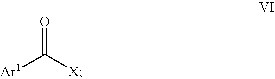
to form a product comprising a compound of formula I 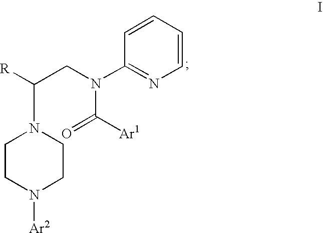
or pharmaceutically acceptable salt thereof;
wherein
R is C₁-C₄ alkyl;
Ar¹ is C₆-C₁₂ aryl optionally substituted with up to three substituents independently selected from the group consisting of halo, alkyl, alkoxy, alkoxycarbonyl, nitro, amino, alkylamino, dialkylamino, haloalkyl, dihaloalkyl, trihaloalkyl, cyano, and amido substituents each having no more than six carbon atoms;
Ar² is dihydrobenzodioxinyl, benzodioxinyl, or phenyl optionally substituted with up to three substituents independently selected from the group consisting of halo, methoxy, halomethyl, dihalomethyl and trihalomethyl;
L is a leaving group; and
X is halo.

2. A process according to claim 1,
wherein said aminoalkyl(pyridine-2-yl)sulfamic acid is 2-aminopropyl(pyridine-2-yl)sulfamic acid.

3. A process according to claim 2,
wherein said aminoalkyl(pyridine-2-yl)sulfamic acid is (2R)-2-aminopropyl(pyridine-2-yl)sulfamic acid.

4. A process according to claim 1,
wherein said aminoalkyl(pyridine-2-yl)sulfamic acid is formed from its 2-(5-alkyl-2,2-dioxo-2λ⁶-[1,2,3]oxathiazolidin-3-yl)-pyridine precursor.

5. A process according to claim 4,
wherein said 2-(5-alkyl-2,2-dioxo-2λ⁶-[1,2,3]oxathiazolidin-3-yl)-pyridine precursor is 2-(5-methyl-2,2-dioxo-2λ⁶-[1,2,3]oxathiazolidin-3-yl)-pyridine precursor.

6. A process according to claim 1,
wherein R is methyl.

7. A process according to claim 1,
wherein Ar¹ is a substituted phenyl.

8. A process according to claim 1,
wherein Ar¹ is a cyano substituted phenyl.

9. A process according to claim 1,
wherein Ar¹ is a p-cyanophenyl.

10. A process according to claim 1,
wherein Ar² is dihydrobenzodioxinyl.

11. A process according to claim 1,
wherein L is halo, tosylate, mesylate, or p-bromophenylsulfonyloxy.

12. A process according to claim 11,
wherein L is mesylate.

13. A process according to claim 11,
wherein L is halo.

14. A process according to claim 13,
wherein L is chloro or bromo.

15. A process according to claim 1,
wherein X is chloro.

16. A process according to claim 1,
wherein said amino-substituted aryl compound is aniline or naphthalenylamine.

17. A process according to claim 1,
wherein said amino-substituted heteroaryl compound is benzodioxane aniline or dihydrobenzodioxane aniline.

18. A process according to claim 17,
wherein said amino-substituted heteroaryl compound is dihydrobenzodioxane aniline.

19. A process according to claim 1,
wherein said tertiary amine is triethylamine.

20. A process according to claim 1,
wherein said 2-halooethanol is 2-chloroethanol.

21. A process according to claim 1,
wherein said dialkylating step is carried out in the presence of at least one inorganic base in aprotic solvent.

22. A process according to claim 1,
wherein said salt of the compound of formula II is an inorganic salt.

23. A process according to claim 22,
wherein said salt of the compound of formula II is a hydrogen chloride salt.

24. A process according to claim 1,
wherein said salt of the compound of formula V is an inorganic salt.

25. A process according to claim 24,
wherein said salt of the compound of formula V is a hydrogen chloride salt.

26. A process according to claim 1,
wherein said activating step is carried at a temperature of from about 0° C. to about 25° C.

27. A process according to claim 26,
wherein said activating step is carried at a temperature of from about 0° C. to about 15° C.

28. A composition according to claim 1,
wherein the compound of formula I is a compound of formula IA:

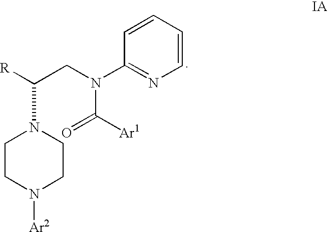

29. A process according to claim 1,
wherein said compound of formula I is 4-cyano-N-{2-[4-(2,3-dihydro-benzo[1,4]dioxin-5-yl)-piperazin-1-yl]-propyl}-N-pyridin-2-yl-benzamide or pharmaceutically acceptable salt thereof.

30. A process according to claim 29,
wherein said compound of formula I is (R)-4-cyano-N-[2-[4-(2,3-dihydro-1,4-benzodioxan-5-yl)-1-piperazinyl-propyl]-N-(2-pyridinyl)-benzamide or pharmaceutically acceptable salt thereof.

31. A product produced by the process of claim 1.

32. A composition, comprising:
the product produced by the process of claim 1; and
at least one pharmaceutically acceptable carrier.

33. A composition, comprising:
at least one compound of formula I: 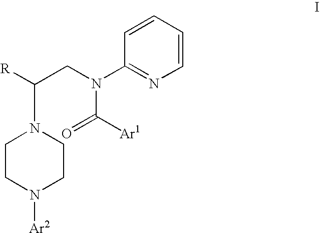
or pharmaceutically acceptable salt thereof;
wherein
R is C₁-C₄ alkyl;
Ar¹ is C₆-C₁₂ aryl optionally substituted with up to three substituents independently selected from the group consisting of halo, alkyl, alkoxy, alkoxycarbonyl, nitro, amino, alkylamino, dialkylamino, haloalkyl, dihaloalkyl, trihaloalkyl, cyano, and amido substituents each having no more than six carbon atoms;
Ar² is dihydrobenzodioxinyl, benzodioxinyl, or phenyl optionally substituted with up to three substituents independently selected from the group consisting of halo, methoxy, halomethyl, dihalomethyl and trihalomethyl;
less than about 0.5% by weight, based on the weight of the composition, of a dimer of the compound of formula I; and
less than about 0.5% by weight, based on the weight of the composition, of a polyalkylation impurity.

34. A compound according to claim 33,
wherein said dimer is a compound of formula A: 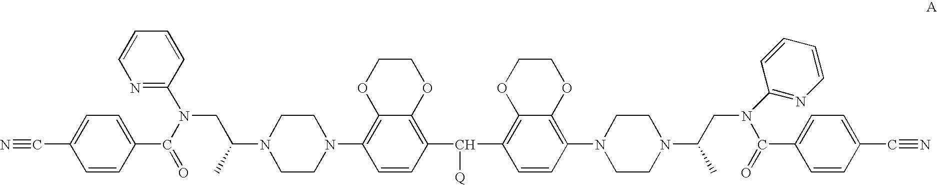
wherein Q is H, —CH₃, —CH(CH₃)₂, CH(CH₂)₂CH₃, or CH(CH₂)₃CH₃.

35. A compound according to claim 34,
wherein Q is H.

36. A compound according to claim 34,
wherein Q is —CH₃, —CH(CH₃)₂, CH(CH₂)₂CH₃, or CH(CH₂)₃CH₃.

37. A composition according to claim 33, further comprising:
at least one pharmaceutically acceptable carrier.

38. A composition according to claim 33,
wherein said compound of formula I is a compound of formula IA: 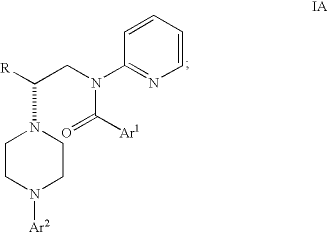
or pharmaceutically acceptable salt thereof.

39. A composition according to claim 33,
wherein R is methyl.

40. A composition according to claim 33,
wherein Ar¹ is a substituted phenyl.

41. A composition according to claim 40,
wherein Ar¹ is a cyano substituted phenyl.

42. A composition according to claim 41,
wherein Ar¹ is a p-cyanophenyl.

43. A composition according to claim 33,
wherein Ar² is dihydrobenzodioxinyl.

44. A composition according to claim 33,
wherein L is a mesylate.

45. A composition according to claim 33,
wherein L is halo.

46. A composition according to claim 45,
wherein L is chloro or bromo.

47. A composition according to claim 33,
wherein said compound of formula I is 4-cyano-N-{2-[4-(2,3-dihydro-benzo[1,4]dioxin-5-yl)-piperazin-1-yl]-propyl}-N-pyridin-2-yl-benzamide or pharmaceutically acceptable salt thereof.

48. A composition according to claim 47,
wherein said compound of formula I is (R)-4-cyano-N-[2-[4-(2,3-dihydro-1,4-benzodioxan-5-yl)-1-piperazinyl-propyl]-N-(2-pyridinyl)-benzamide or pharmaceutically acceptable salt thereof.

49. A composition according to claim 33,
wherein said dimer is present at a level of less than about 0.2% by weight, based on the weight of the composition.

50. A composition according to claim 49,
wherein said dimer is present at a level of less than about 0.1% by weight, based on the weight of the composition.

51. A composition according to claim 50,
wherein said composition is substantially fee of said dimer.
